(19) United States
(12) Patent Application Publication
(10) Pub. No.: US 2016/0314263 A1
(43) Pub. Date: Oct. 27, 2016

(54) METHOD AND SYSTEM FOR MONITORING EXCREMENT DATA

(71) Applicant: SCA HYGIENE PRODUCTS AB, Göteborg (SE)

(72) Inventors: Carolyn BERLAND, Mölndal (SE); Mattias BOSAEUS, Kallered (SE)

(73) Assignee: SCA Hygiene Products AB, Goteborg (SE)

(21) Appl. No.: 15/104,296

(22) PCT Filed: Dec. 20, 2013
(86) PCT No.: PCT/SE2013/051597

§ 371 Date: Jun. 14, 2016

(51) Int. Cl. G06F 19/00; A61F 13/42

(52) U.S. Cl. CPC G06F 19/3418; A61F 13/42; A61F 2013/424

(57) ABSTRACT

A method, processing unit, and system for monitoring excrement data for seeking a pattern for a monitoring period includes acquiring excrement data including excrement data for at least two different time periods, each substantially corresponding to the monitoring period for which a pattern of events is sought. The excrement data is associated with at least one absorbent article and a person wearing said at least one absorbent article and includes information of excrement supply into an absorbent article in form of excrement events. Each excrement event has an associated time mark or associated time information from which an associated time mark may be derived. Further, the method includes providing a series of sequential non-overlapping time intervals, together substantially extending over the monitoring period, associating each time interval with a segment of a diagram, calculating a number of excrement events corresponding to each time interval for all time periods for which data is acquired, and associating the number of excrement events corresponding to each time interval with a corresponding segment of the diagram.

### CROSS-REFERENCE TO PRIOR APPLICATION

This application is a §371 National Stage Application of PCT International Application No. PCT/SE2013/051597 filed Dec. 20, 2013, which is incorporated herein in its entirety.

### TECHNICAL FIELD

Generally, the disclosure relates to a method for monitoring excrement data for a period of time. The excrement data is associated with at least one absorbent article and a person wearing said at least one absorbent article. Further, the disclosure relates to a system adapted to perform a method for evaluating excrement data.

### TECHNICAL BACKGROUND

Systems for monitoring incontinence are known in the art. For instance, WO96/14813 A1 discloses an incontinence monitoring system and is particularly concerned with a system for the detection monitoring and management of urinary, faecal and other forms of incontinence. The system includes a plurality of sensors and a monitor to receive and record signals from the sensors, each sensor being adapted to be associated with a respective person and being responsive to urinary and/or faecal incontinence in that person. The monitor is capable of recording the time of onset of each incontinence condition and of indicating any regularity or pattern of incontinence in each said person. Over a period of time, for example 1 or 2 weeks, any pattern or regularity of incontinence periods for a particular patient may be identified from the recorded information, either manually or using appropriate software, and the patient, or nursing staff, may be arranged to anticipate an oncoming period of incontinence.

However, a drawback of the system disclosed in WO96/14813 A1 may be that the information recorded by the system may be difficult to interpret for identifying a pattern or regularity of incontinence periods for a particular patient. If the pattern or regularity is identified manually, the skills of the person identifying the pattern may affect the result. Further, such manual identification gives an arbitrary result. Still further, interpretation of complex data may be time consuming and the time available for the interpretation may affect the result.

Another system and method for accurately predicting approximate times an incontinent person without intervention would void in the future is disclosed in U.S. Pat. No. 5,416,469. A temperature sensor, which is connected to a sampling/recording meter, is imbedded in a diaper. The meter measures and records and stores the temperature of the sensor at time intervals of, for example, one minute over a predetermined period of, for example, three days. Raw time and temperature data are downloaded from meter to computer. The raw temperature data are in the form of digital electrical signals characteristic of sensor and meter. Raw time data are represented by the intervals between the raw temperature data. The, raw time data are correlated with a predetermined data sample period. The raw time data are correlated with the data sample period by aligning the beginning of the sequence of raw temperature data with the corresponding time in the data sample period. A voiding event cluster time period is selected by the operator. Then, the selected voiding event cluster time period is applied to the beginning of the data sample period including data acquired of multiple sampling periods. Thereafter, the data designated as voiding events are scanned and counted to identify the number of voiding events during the selected voiding event cluster time period. The number of voiding events occurring within the selected voiding event cluster time period is recorded. The selected voiding event cluster time period is shifted by a preselected incremental interval (e.g., 5 minutes) within the data sample period. The record of numbers of voiding events identified during the incrementally adjusted selected voiding event cluster time periods is reviewed to identify maximums in the values of recorded numbers, the maximums corresponding to clusters of voiding events. Typically, the maximums will extend over several successive selected voiding event cluster time periods and will correspond to a time range during which voiding events may be expected. It will be appreciated that decreasing the voiding event cluster time period could provide enhanced resolution in the determination of such time ranges. The computer renders a display indicative of the time ranges during which voiding events may be expected. The display may be rendered graphically or as a text explanation.

However, a drawback of the system disclosed in U.S. Pat. No. 5,416,469 may be that although the information is easier to interpret than that recorded by the system of WO96/14813 A1, the method requires that the person using the system has a certain level of knowledge for choosing correct voiding event cluster time periods and for interpreting the resulting data. Further, the resulting data appears relatively complex, and interpretation of complex data may be time consuming and the time available for the interpretation may affect the result. Still further, the method appears complicated.

Therefore, there is still a need of improved methods and systems that at least alleviates the problems of prior art systems and offers identification of regularities or patterns from the recorded data such that the risk of misinterpretation as well as time consumed for analysis of the collected data is reduced. Further, there is a need of simple improved methods and systems for identifying such liquid discharge event patterns, which eliminates the dependency of the analysis result of knowledge of a person analysing recorded data as well as time available for analysis of recorded data.

### SUMMARY

The present disclosure is based on an insight that by arranging excrement data into time intervals associated with a segment of a diagram or a time diagram, a pattern for excrement events may be easily interpreted by a person using the system. Thus, the pattern, and consequently also a toileting schedule, may be found accurately independently of the level of knowledge of the person using the system as well as time available for analysis of data. In such a way arbitrariness during analysis is eliminated and time used for analysis is decreased.

The method for monitoring or evaluating excrement data for seeking a pattern for a monitoring period includes acquiring excrement data including excrement data for at least two different time periods, each substantially corresponding to the monitoring period for which a pattern of events is sought. The excrement data is associated with at least one absorbent article and a person wearing said at least one absorbent article and includes information of excrement supply into an absorbent article in form of excrement events. Each excrement event has an associated time mark or associated time information from which an associated time mark may be derived. Further, the method includes providing a series of sequential non-overlapping time intervals, together substantially extending over the monitoring period and associating each time interval with a segment of a diagram. The method includes also calculating a number of excrement events corresponding to each time interval for all time periods for which data is acquired and associating the number of excrement events corresponding to each time interval with a corresponding segment of the diagram.

The monitoring period, for which a pattern of events is sought, may be any period for which an event pattern is sought, for instance a 24 hour day, a period during which the person wearing said at least one absorbent article is awake, a shift etc. The excrement data may be acquired provided for a period which is continuous or discontinuous and includes the at least two different time periods according to the claims. The different time periods may originate from any different/separate days or shifts, such as subsequent days or days with one or several days in between as far as the incontinence condition of the person wearing the absorbent article is not significantly changed. In all examples above, the different time periods of data that corresponds to the monitoring period is intended to mean that the period corresponds to approximately same hours (or minutes) of day.

The excrement data is considered to be any data indicative of supplies of excrement into the absorbent article. Such supplies of excrement received by the absorbent article due to an excrement event, or supply of excrement event, may be detected using suitable sensors arranged in the absorbent article. The excrement may be in form of faeces, urine, or a mixture thereof. The data is collected or acquired using an absorbent article including a sensor. The sensor may be any suitable sensor, such as a liquid discharge sensor, a gas sensor, a temperature sensor, as long as the sensor is able to detect a supply of excrement into an absorbent article.

If the sensor associated with the absorbent article includes several sensor zones or sensor segments, or if several sensors are arranged in the absorbent article, an excrement event is considered to have occurred when after a substantially constant property or state of the absorbent article, a further sensor segment, sensor zone, or sensor indicates that the portion of the absorbent article where that sensor segment, sensor zone, or sensor is arranged has received supply of excrement, i.e. has changed the property or the absorbent state. Since the property or state of the article was substantially constant before the further sensor segment, zone or sensor indicated a change, a second or further excrement event, leading to the liquid of the excrement to spread the article to a further sensor zone, sensor segment, or sensor, is considered to have occurred.

The excrement events may be detected prior to the acquiring providing data. The excrement data may include information of excrement events in any suitable form, such as in form of a vector including merely time marks for each detected or occurred event, in form of a matrix including both time marks in one column and some kind of indication for each detected event in another column, such as symbols or numbers 0 (for non-events) and 1 (indicating events), respectively. The time marks may include information in form of minutes, in form of hours and minutes irrespective of the day, or in form of days, hours, and minutes.

Herein, absorbent article refers to an adult incontinence product, a baby or toddler diapers, sanitary towels, liners or other known absorbent articles. The diagram or time diagram of the claims may be any suitable diagram.

According to at least one example embodiment, the method may further include combining the excrement data of the at least two different time periods to aggregated data extending over the monitoring period, covering the data for the at least two different time periods.

According to at least one example embodiment, combining the data to excrement data of the at least two different time periods to aggregated data may include superposing the excrement data of the at least two different time periods such that each event of the superposed excrement data is distinguishable and such that the hours and/or times of the superposed sets of data coincide with the hours and/or times of the monitoring period.

Thus, for such superposed data the data of the at least two different time periods is aligned with regard to time with the monitoring period.

The number of excrement events associated with each time interval, or the frequency of the excrement events for each time interval, may be calculated either for the aggregated data, or for each of the at least two different time periods separately. However, it is advantageous to calculate the sum for the aggregated data or independently of from which of the at least two time periods corresponding to the monitoring period the excrement events originate form, since it allows for higher probability for reoccurrence. In such a case, the estimation of the pattern of excrement events is based on a larger set of excrement data, which results in higher certainty.

According to at least one example embodiment, the excrement data may be in form of absorbent state data and an excrement event is indicated by a change of the absorbent state of the absorbent article.

An absorbent state is intended to mean any state indicative of the amount absorbed by the absorbent article. Thus, also an absorbent article which has not yet absorbed any supplies of excrement is considered to have an absorbent state. A change in an absorbent state of an absorbent article is intended to mean any change of the absorbent state, from a first substantially stable absorbent state to any other substantially stable absorbent state. Herein, a stable state is intended to mean a state which is substantially constant during a period of time, such as at least a period having the length of a time interval of the series of time intervals together extending over the monitoring period. A change of absorbent state may occur due to supply of excrement into the absorbent article, that is, an excrement event. A change from an absorbent state being indicative of a dry article into an absorbent state being indicative of an article which has received a supply if excrement is also considered to be a change of absorbent state. The absorbent state of the article may be indicated by an electrical or other property of the absorbent article.

According to at least one example embodiment, the excrement data may be in form of electrical property data and an excrement event is indicated by a change of the electrical property of the absorbent article.

According to at least one example embodiment, the excrement data may be in form of absorbed excrement volume data and an excrement event is indicated by a change of the absorbed excrement volume of the absorbent article.

According to at least one example embodiment, calculating the number of excrement events corresponding to each time interval may include calculating a sum of excrement events corresponding to each time interval, wherein the sum is a sum of excrement events corresponding to each time interval originating from any of the at least two different time periods, corresponding to the monitoring period. Further, associating the number of excrement events corresponding to each time interval with a corresponding segment of said diagram may include associating the sum of excrement events with a corresponding segment of said diagram.

Such a sum of excrement events of the aggregated data is a total sum of supply of excrement for the at least two different time periods corresponding to the monitoring period for which data is acquired.

According to at least one example embodiment, the method may further include correlating the time mark of each excrement event with a corresponding time interval prior to calculating the number of excrement events corresponding to each time interval.

That is, if there is for instance an excrement event having a time mark 06.03 originating from the first time period for which excrement data is acquired and another time excrement event having a time mark 06.15 originating form the second period for which excrement data is acquired, and if one of the time intervals is between 06.00 and 07.00, then the sum of excrement events corresponding to this time interval is 2.

According to at least one example embodiment, the time intervals may be pre-determined, and have may be equal lengths.

Herein, a predetermined series of time intervals is intended to be predetermined in respect of the number of time intervals, the length of the time intervals, the sum of the length of the time intervals, or the alignment of the time series in relation to the monitoring period, etc.

According to at least an exemplary embodiment, the time intervals may be non-selectable by the user or care giver.

Herein, non-selectable is intended to mean either predetermined or determined by the system. That is, a person using the system, i.e. an operator or nurse, cannot select the length of the time intervals. Alternatively, the time intervals may be preselected or selected by the system

According an alternative exemplary embodiment, the series of time intervals may be determined by the method or the system adapted to perform the method.

According to at least an exemplary embodiment, a sum of the length of said time intervals may be shorter than the period of time corresponding to the period for which data is acquired or provided; the sum of the length of said time intervals may equal the length of the monitoring period.

According to at least one example embodiment, the length of each of said predetermined time intervals may be 20 minutes to 150 minutes, may be 30 to 90 minutes, may be 45 to 75 minutes, or may be 55 to 65 minutes.

The length of the intervals is based on research for finding an interval length which is suitable for monitoring a pattern of excrement events. The inventors have found that especially a length of approximately 1 hour is advantageous, since it allows for a reliable visualisation of a pattern of excrement events while limiting the impact of noise satisfyingly.

According to at least one example embodiment, the total measurement period, including the at least two different time periods for which excrement data is acquired or provided, may be predetermined, and may have a length of two to seven times the monitoring period, may have a length of at least three times the monitoring period, and may have substantially a length of three times the monitoring period.

That is, the different time periods for which data is provided or acquired, form together a measurement period which is either continuous or discontinuous. The measurement period covers 2-7 monitoring periods or may be at least 3 monitoring periods, or may be substantially 3 monitoring periods.

According to at least one example embodiment, the number of associated excrement events for each time interval may be associated with a correlating graphical scheme applied to the segments of said diagram.

The graphical scheme may for instance be in form of rectangles having different heights or widths, wherein a wider or higher rectangle is associated with a higher number of excrement events. Other alternatives are a predetermined colour gradient wherein a lighter colour is associated with a lower number and a darker colour with a higher number, other forms than rectangles, such as circles or triangles having different sizes or colours or different colour densities, etc. or any other suitable graphical scheme which makes the pattern easy to perceive.

According to at least one example embodiment, the number of associated excrement events for each time interval may be associated with a correlating colour scheme applied to the segments of said diagram.

The colour scheme may be in form of a colour gradient or different densities for one colour, or different colours for different number of events, etc.

According to at least one example embodiment, the colour scheme may be in form of a colour gradient, and may be correlating an increasing darkness with an increasing number of associated excrement events.

According to at least one example embodiment, toileting of the person associated with the excrement data may be recommended during a time interval prior to an interval for which the number of associated excrement events is equal to or larger than the number of to the monitoring periods corresponding time periods for which data is acquired.

According to at least one example embodiment, the time interval for which toileting is recommended may be indicated in the diagram by a predetermined symbol.

According to at least one example embodiment, the number of associated excrement events for each time interval may be arranged in form of a linear diagram.

According to at least one example embodiment, the number of associated excrement events for each time interval may be arranged in form of a circumferential diagram extending over the monitoring period, wherein each segment of the circumferential diagram corresponds to each of the time intervals.

Herein, a circumferential diagram or time diagram is intended to mean a time diagram which has a circumferential form, wherein the start time and the end time coincide, such as a circular diagram, a 24 hour clock diagram, a quadratic 24 hour clock diagram or any other time diagram having a circumferential form suitable for illustrating the information.

According to at least one example embodiment, the number of associated excrement events for each time interval may be arranged in form of a circular diagram extending substantially over the monitoring period, wherein each segment of the circular diagram corresponds to each of the time intervals.

According to at least one example embodiment, the circumferential diagram may be in form of a circular diagram.

According to at least one example embodiment, each segment of the circumferential diagram may correspond to 55 to 65 minutes.

According to at least one example embodiment, the method may further include visually representing the diagram for visualising a pattern of events based on the acquired data.

A second aspect relates to a processing unit adapted to monitor or evaluate excrement supply data. The processing unit is adapted to process an output signal generated by a sensor arranged in an absorbent article. The processing unit is adapted to acquire excrement data including excrement data for at least two different time periods, each substantially corresponding to the monitoring period for which a pattern of events is sought. The excrement data includes information of excrement supply into an absorbent article in form of supply excrement of events and each excrement event has an associated time mark or associated time information from which an associated time mark may be derived. The processing unit is adapted to provide a series of sequential non-overlapping time intervals, together substantially extending over the monitoring period, and associate each time interval with a segment of a diagram or time diagram, which diagram substantially extends over the monitoring period. The processing unit is adapted to calculate a number of excrement events corresponding to each time interval, and arrange said number of excrement events corresponding to each time interval into a corresponding segment of said diagram.

The sensor may be any suitable sensor, such as a liquid discharge sensor, a gas sensor, a temperature sensor, as long as the sensor is adapted to detect a property of the absorbent article which changes due to a supply of excrement. From the change of such a property, an excrement event may be derived. Such a property may be an electrical property, such as impedance, resistance or similar, or an absorbent state of the absorbent article. The sensor may be reusable and adapted to be attached and detached to the absorbent article or the sensor may be a disposable sensor intended to be used once and arranged inaccessibly within the absorbent article. Herein, the term “inaccessibly” means that the sensor is not accessible to the user without breaking the absorbent article at least partly.

In an exemplary embodiment, the supply of excrement into the absorbent article may be detected based on data relating to the property of the absorbent article acquired by the sensor associated with the absorbent article prior to associating or correlating each time mark of each excrement event with a corresponding time interval.

According to at least one example embodiment, the processing unit may be further adapted to calculate the number of excrement events corresponding to each time interval by calculating a sum of excrement events originating from each period, corresponding to the monitoring period, corresponding to each time interval over the total period for which data is acquired.

According to at least one example embodiment, the processing unit may be further adapted to correlate the time mark of each excrement event with a corresponding time interval prior to calculating the number of excrement events corresponding to each time interval.

According to at least one example embodiment, the processing unit may be further adapted to associate a number of associated excrement events for each time interval with a graphical scheme and to apply said scheme to the segments of said diagram.

According to at least one example embodiment, the processing unit may be adapted to perform the method according to the first aspect.

The example embodiments of the processing unit have similar features and advantages as the method according to the first aspect.

A third aspect relates to a system for monitoring excrement data. The system includes a sensor associated with an absorbent article, which is arranged to generate an output signal representative of the excrement state of said absorbent article over at least two different time periods, and a processing unit adapted to perform the method according to the first aspect.

Herein, excrement state is intended to mean a state or property of the absorbent article indicative of occurred excrement events, if any. Such a system is advantageous, since it allows for more accurate monitoring or evaluation of excrement data. Other advantages with such a system are similar to the advantages of the first aspect described above.

According to at least one example embodiment, the system may further include output means for visually representing the number of correlated time marks of excrement events for each corresponding time interval for visualising a pattern of events based on the excrement data.

By visually representing the number of correlated time marks for each corresponding time interval a pattern of events may be visualised and from that pattern a recommended toileting scheme may be established.

According to at least one example embodiment, the system may further include means for visually representing the diagram or time diagram illustrating a pattern of excrement events or a toileting schedule.

According to at least one example embodiment, the system may further include means for recommending toileting a time interval or segment prior to a segment for which the number of events is equal to or larger than the number of monitoring periods for which data is acquired. If desired, recommended toileting intervals may be indicated in the diagram using a suitable symbol.

The example embodiments of the system have similar features and advantages as the method according to the first aspect.

Generally, all terms used in the claims are to be interpreted according to their ordinary meaning in the technical field, unless explicitly defined otherwise herein. All references to “a/an/the element, device, component, means, step, etc” are to be interpreted openly as referring to at least one instance of said element, device, component, means, step, etc., unless explicitly stated otherwise.

Other objectives, features and advantages of embodiments of the present invention will appear from the following detailed disclosure, from the attached dependent claims as well as from the drawings.

### BRIEF DESCRIPTION OF THE DRAWINGS

These and other aspects of the present disclosure will now be described in more detail, with reference to the appended drawings showing embodiment(s) thereof, in which:

FIG. 1 is a flow chart illustrating schematically a method for monitoring excrement data according to at least an exemplary embodiment of one aspect of the present disclosure;

FIG. 2 is an example of excrement data obtained for an absorbent article during a measurement period of 7 days;

FIG. 3 is an example of excrement data acquired for an absorbent article during a period of 7 days monitored according to an exemplary embodiment of the disclosure;

FIG. 4 is a flow chart illustrating schematically a method for monitoring excrement data according to at least an exemplary embodiment of one aspect of the present disclosure;

FIG. 5 is an example of a smoothing function according to an exemplary embodiment;

FIG. 6 is an example of excrement data monitored according to an exemplary embodiment of the present disclosure;

FIG. 7 is an alternative example of excrement data monitored according to an exemplary embodiment of the method of the present disclosure;

FIG. 8 is an alternative example of excrement data monitored according to an exemplary embodiment of the method of the present disclosure;

FIG. 9 is an alternative example of excrement data monitored according to an exemplary embodiment of the method of the present disclosure; and

FIG. 10 illustrates a system for monitoring excrement data according to at least a first exemplary embodiment of the second aspect of the present disclosure.

All the figures are highly schematic, not necessarily to scale, and they show only parts which are necessary in order to elucidate the invention, other parts being omitted or merely suggested.

### DETAILED DESCRIPTION OF PARTICULAR EMBODIMENTS

The invention will now, by way of example, be described in more detail by means of embodiments and with reference to the accompanying drawings, but is not limited thereto.

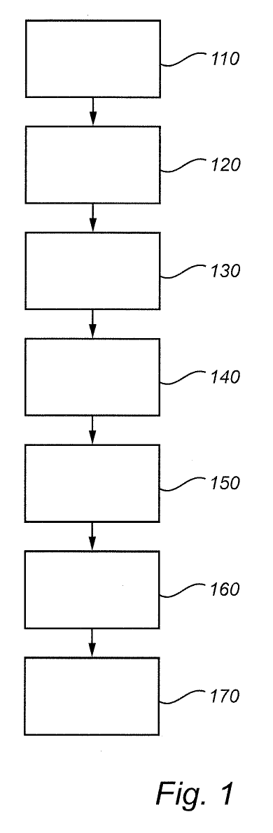

FIG. 1 illustrates a method for evaluating or analysing excrement data for seeking a pattern for a monitoring period. The excrement data is associated both with at least one absorbent article and with a person wearing said at least one absorbent article. The method includes the steps of:
acquiring excrement data including including excrement data 110 for at least two different time periods, each substantially corresponding to the monitoring period for which a pattern of events is sought;
providing a series of sequential non-overlapping time intervals 120, extending substantially over the monitoring period; and associating the time mark associated with each excrement event with a corresponding time interval 130.

The excrement data includes information of excrement supply into an absorbent article in form of excrement events. Each excrement event has an associated time mark or associated time information from which an associated time mark may be derived. By providing a series of sequential non-overlapping time intervals extending substantially over the monitoring period, the monitoring period may be divided into a series of time intervals. Therefore, the length of said time intervals is shorter than said period of time for which data is acquired or provided and a sum of the length of said time intervals substantially equals the length of the monitoring period. The monitoring period, for which a pattern of events is sought, may be any period for which an event pattern is sought, for instance a 24 hour day, a period during which the person wearing said at least one absorbent article is awake, a shift at a nursing home, etc. The excrement data may be provided for a measurement period which is continuous or discontinuous. The different time periods may originate from any different/separate days or shifts, such as subsequent days or days with one or several days in between as far as the incontinence condition of the person wearing the absorbent article is not significantly changed. For all examples above, the time periods of data that corresponds to the monitoring period is intended to correspond substantially to same hours (or minutes) of day.

The excrement data may be in form of absorbent state data and an excrement event is indicated by a change of the absorbent state of the absorbent article. An absorbent state is intended to mean any state indicative of the amount absorbed by the absorbent article. Thus, also an absorbent article which has not yet absorbed any supplies of excrement is considered to have an absorbent state. A change in an absorbent state of an absorbent article is intended to mean any change of the absorbent state, from a first substantially stable absorbent state to any other substantially stable absorbent state. Herein, a stable state is intended to mean a state which is substantially constant during a period of time, such as at least a period having the length of an interval of the series of time intervals together extending over and covering the monitoring. A change of absorbent state may occur due to supply of excrement into the absorbent article, that is, an excrement event. A change from an absorbent state being indicative of a dry article into an absorbent state being indicative of an article which has received a supply of excrement is also considered to be a change of absorbent state.

Alternatively, the excrement data is in form of electrical property data and an excrement event is indicated by a change of the electrical property of the absorbent article. The excrement data includes information of excrement events in form of a vector or matrix including merely time marks for each detected or occurred event. The time marks include information for the actual times when an excrement event has occurred, is detected, or is considered to have occurred in form of days, hours, and minutes. The exemplary methods may be applied to excrement data of different types. For instance, the time marks of the excrement data may be alternatively arranged as a matrix including day or date in one column, hour in another column and minutes in a third column, which are associated with each other.

The data in form of days, hours and minutes, may be arranged to subgroups irrespective of the day. By disregarding the day the data is superposed without losing any information of each separate excrement event.

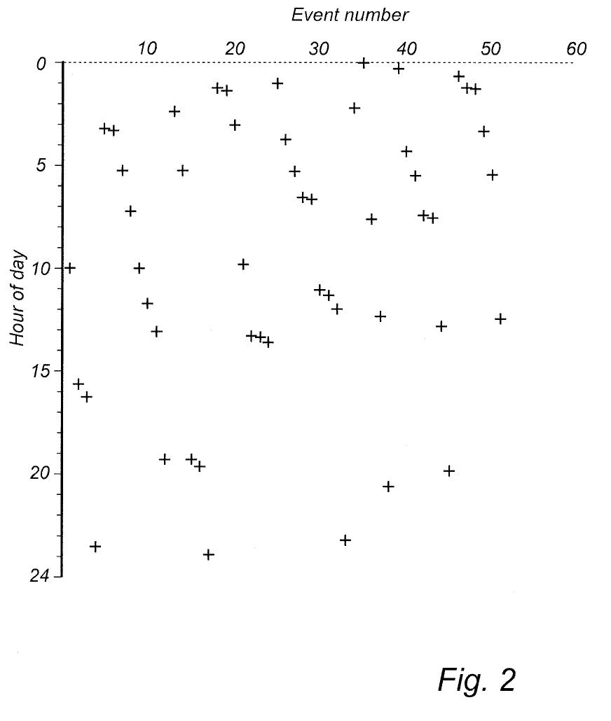

FIG. 2 shows excrement data acquired for a total measurement period of seven days and for a monitoring period of a 24 hour day. The time is on y axis and the number of void is in chronological order on x axis. The excrement data is plotted at the actual time of the events, that is, not associated or correlated with an interval into which the monitoring period (in this case a 24 hour day) is divided. It is clear from FIG. 2 that a pattern of events is relatively difficult to identify quickly based on a graph of unanalysed data.

Alternatively, each time mark may include information for the actual times when an excrement event has occurred, is detected, or is considered to have occurred in form of minutes irrespective of the day. In such a case, each time mark for the first time period of the at least two different periods, or the first day, includes the time between the beginning of the measurement period and the point of time each excrement event is considered to have occurred, or is detected, in minutes. For the second time period each time mark in such a case includes the length of the first time period in minutes and the time between the beginning of the second period and the point of time each the excrement event is considered to have occurred, or is detected, in minutes, that is, the number of minutes from the beginning of the second period until each excrement event is considered to have occurred or is detected. From this type of data, time marks for excrement events of each time period may be obtained by subtracting the length of each time period in minutes from each time mark originating from each subsequent time period after the first, i.e. the second, third, etc. In this way the data for each different measurement period may be superposed or added together without losing any information of occurred or detected excrement events. Thereafter, the time marks of the excrement events may be associated with the time intervals into which each monitoring period is divided into, i.e. the series of time intervals.

Still alternatively, the time marks may include information in form of hours and minutes irrespective of the day. In such a case the time marks are similar to those as described for minutes above, but in form of hours and minutes. Thereafter, the data is handled similarly as is described above.

The excrement data may include information of excrement events in any suitable form, such as raw data for change in electrical property, in form of a vector including merely time marks for each detected or occurred change. Alternatively, excrement data may include information in form of a matrix including both time marks in one column and some kind of indication for each detected change in another column, such as symbols or numbers 0 (for non-changes) and 1 (indicating changes), respectively. The time marks may include information in form of minutes, in form of hours and minutes irrespective of the day, or in form of days, hours, and minutes.

The association of time interval may mean association in form of correlation to a time interval which is relatively shorter than the monitoring period, or association with a bandwidth of a Kernel smoothing applied to the time marks, or any other similar association with a time period.

The excrement may be in form of faeces, urine, or a mixture thereof. The data may include data for both supply of faeces events and supply of urine events, which events might be discriminated from each other, if desired, a combination of such events, or only one type of events. Data for different types of events may be analysed together or separately.

In the exemplary embodiment in FIG. 1, the excrement events are detected based on data acquired by a sensor associated with the absorbent article prior to associating or correlating each time mark of each excrement event with a corresponding time interval. The excrement events may be automatically detected by the system. The sensor may be any suitable sensor, such as a liquid discharge sensor, a gas sensor, a temperature sensor, as long as the sensor is adapted to detect a supply of excrement into the absorbent article.

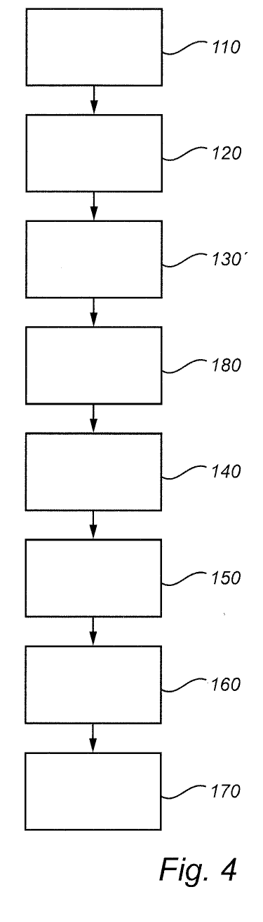

In the exemplary embodiment in FIG. 4, associating the time mark associated with each excrement event with the corresponding time interval is in form of correlating 130′ the time mark of each excrement event with the corresponding time interval independently of the time period to which said excrement event corresponds or is associated with.

The method illustrated in FIG. 4, further includes arranging the excrement data into subsets 140 based on the correlation of each time mark with the corresponding time interval of said first series of intervals over time. Such arrangement results in subsets of data in form of combined data for all different time periods. In other words, arranging the data into subsets 140 is a way of combining data to subsets of data corresponding to each time interval. A number of events for each time interval may be obtained by calculating the number of events corresponding to each subset. In this way a pattern over a number of time periods, such as days, corresponding to the monitoring period may be obtained. From such a pattern, time intervals for recurring excrement events may be obtained.

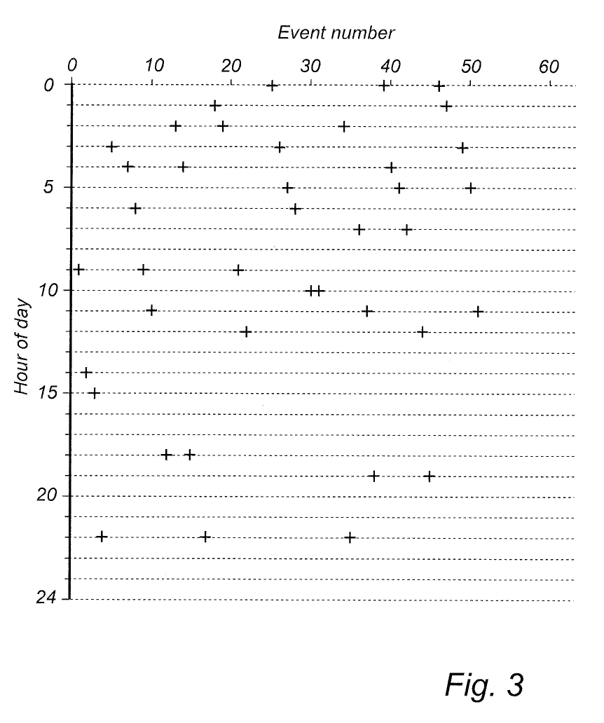

The data arranged to subsets is associated or correlated with a corresponding time interval of the provided series of sequential non-overlapping time intervals, which divide the monitoring into intervals. FIG. 3 shows excrement data for three different time periods, here three days, and for a monitoring period of one 24 hour day. In FIG. 3 time in hours is on y axis and the event number on x axis. The events are numbered in chronological order and the first event is assigned number 1. For the embodiment in FIG. 3 the non-overlapping sequential time intervals have a length of 1 hour and extend between: 00 and 01, 01 and 02, 02 and 03, 03 and 04 etc. Thus, in FIG. 3 the data is associated or correlated to time intervals having a length of one hour. Thereafter, the number of excrement events for each time interval may be determined. For instance, for excrement data shown in FIG. 3, the number of time events associated with the time interval between 11 and 12 is 3.

Alternatively, the data may first be associated with the time intervals which together extend over the monitoring period, or into which a monitoring period is divided, and thereafter the data relating to different measurement periods may be superposed.

The method further includes calculating the number of events 150 associated or correlated with each time interval and estimating a pattern 160 of excrement events for the person associated with the absorbent article is estimated based on the calculation of the number of events associated/correlated with each time interval.

In the exemplary embodiment in FIG. 1, the number of events associated or correlated with a time interval, which is equal to or larger than the number of different time periods, corresponding to the monitoring period, for which data is provided or acquired is indicative of a recurring excrement event. The method in FIG. 1 further includes (automatically) recommending toileting 170 of the person associated with the data during the time interval prior to a time interval which is associated with a number of events which is equal to or larger than the number of different time periods, corresponding to the monitoring period, for which data is provided. Such a recommendation provides the care giver automatically a toileting interval during which it is advantageous to take the person associated with the absorbent article, i.e. the care taker to the toilet. A recommended time interval for toileting is more advantageous than a recommended specific time, since it makes it easier for the care giver to plan their work. The recommendation may be in form of a printed list of recommended toileting time intervals or in form of a diagram with graphical indications for recommended toileting intervals.

In the embodiment in FIG. 1, the series of sequential time intervals are non-selectable by a user and/or the time intervals have non-selectable lengths. Further, the lengths of the time intervals can be equal. That is, a person using the system, i.e. an operator or a nurse, cannot select the length of the time intervals. The predetermined series of time intervals are predetermined in respect of the number of time intervals, the length of the time intervals, the sum of the length of the time intervals, and/or the alignment of the time intervals in relation to the monitoring period, etc. According to an alternative embodiment, the series of sequential time intervals are determined or preselected by the method or by the system adapted to perform the method.

The length of each of said predetermined time intervals is 20 minutes to 150 minutes, 30 to 90 minutes, 45 to 75 minutes, or 55 to 65 minutes. The total period of time for which data is acquired or provided, including the at least two different time periods of the claims, is pre-determined, and can have a length of two to fourteen times the length of the monitoring period for which a pattern of events is sought, a length of at least three times the length of the monitoring period, or substantially a length of three times the length of the monitoring period. That is, for being able to ensure that the found patter of events is reliable, the excrement data includes data for at least a total period covering two to fourteen different periods corresponding to the monitoring period. In particular embodiments, the inventors found that data for a total period covering three different time periods is a good compromise between the reliability of the pattern and need and cost for measurements. The total period of time for which data is provided may be continuous or discontinuous. If the total period is discontinuous, the length of the period for which data is provided is considered to be a sum of the partial periods, which are separated from each other by periods for which data is not provided.

Further, the absorbent article includes a sensor, and said excrement data is collected or acquired using the sensor. The sensor is described more in detail below.

According to the embodiment in FIG. 4, the method further includes smoothing each time mark associated to the excrement events. Smoothing is performed by applying a Kernel smoothing method. That, is a Kernel function is applied to each time mark, after associating each time mark with a corresponding time interval. Alternatively, the smoothing may be performed before associating each time mark with a corresponding time interval.

During smoothing, each time mark is associated with a Kernel function or a scaled Kernel function. The association is by applying a Kernel function to each time mark. The Kernel function has a predetermined arbitrary height corresponding to a single excrement event, and suitable bandwidth, h, as defined by standard deviation of the Kernel function, of the Kernel function is chosen.

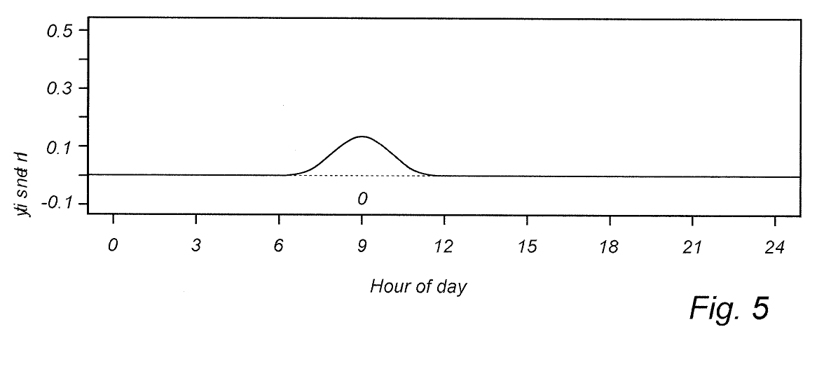

FIG. 5 shows a time mark for an excrement event at 9 am and a Kernel function applied to the event. As is obvious from FIG. 5, the Kernel function is symmetric around each time mark. That is, the time mark is at the centre of a symmetric function having equal extension in time before as well as after the time mark. The Kernel function in FIG. 5 is in form of a Gaussian function. Since the Kernel function is in form of a Gaussian function, the bandwidth is the standard deviation of the Gaussian function.

Generally, the Gaussian function, K, may be expressed as:

$$K ( t ) = \frac{1}{\sqrt{2 π}} {e}^{- \frac{1}{2} {t}^{2}}$$

Generally, a scaled Gaussian function, Kₕ may be expressed as:

$${K}_{h} ( t ) = \frac{1}{\sqrt{2 {πσ}^{2}}} {e}^{- \frac{1}{2} {( \frac{t}{σ} )}^{2}}$$

By applying a symmetric Kernel function to each time mark, the time marks of each event is assigned an extension in time, instead of being a single point of time. Thus, a time mark for an excrement event slightly after a specific time interval may be linked to a group of time marks associated with the specific time interval relatively simply and efficiently. Alternatively, an excrement event slightly before a specific time interval may be linked to a group of excrement events associated with the specific adjacent later time interval.

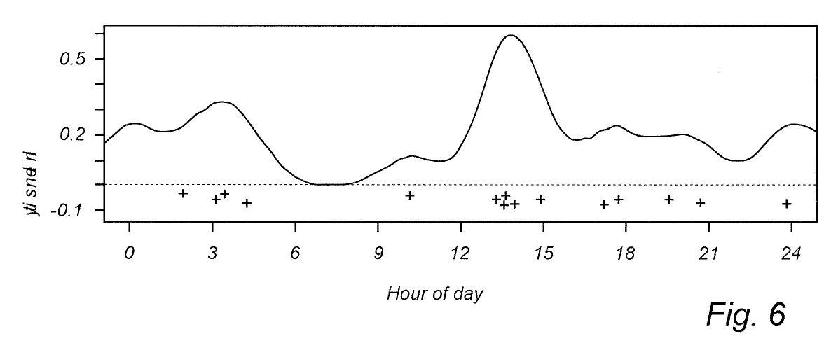

FIG. 6 discloses excrement data analysed according to at least an embodiment of the inventive method for a total measurement period of three 24 hour days. For the excrement data in FIG. 6 the monitoring period is a 24 hour day. The excrement data for the three 24 hour days is first arranged to subgroups and thereafter correlated with non-overlapping sequential time intervals having a length of one hour and beginning at 00.00 am, i.e. midnight, plotted in the FIG. 6 as plus symbols below zero. Thereafter, the excrement data is smoothed by applying a Kernel function in form of a Gaussian function having a height approximately 0.1 and a bandwidth of approximately 1 hour is applied to each time mark of the excrement supply data. The smoothed excrement data is plotted as a continuous curve in FIG. 6. From such a continuous curve peaks having a value equal to or higher than n*k, where n is the number of time periods, corresponding to the monitoring period, for which data is provided, and k is the predetermined height of the kernel function applied to each time mark, may be identified. Since the excrement events are associated with or correlated to a corresponding time interval, the peaks may as well be associated with or correlated to a corresponding time interval. In addition to including information for all the excrement events associated with each time interval itself, the peaks will include information for excrement events adjacent to each time interval. Depending on the chosen bandwidth the influence of excrement events within one time interval in relation to another adjacent time interval may be influenced.

Further, all peaks and their corresponding time marks may be identified and thereafter divided by k, the height of the kernel function. Thus, the number of excrement events corresponding to the identified peaks and their time marks may be calculated. Such information may be visually represented.

Still alternatively, if the excrement events are associated with or correlated to a corresponding time interval prior to smoothing and the exact time marks are smoothed, such information may be used for identifying time intervals corresponding to the peaks.

From FIG. 6 it is clear that for instance the four events having time marks around 13 and 14 may be linked to a single group instead of one of them being a single event in another interval than the three others.

The inventors have found that a suitable bandwidth h of the Kernel function is 20-150 minutes, 30-90 minutes, 45-75 minutes, or 55-65 minutes. In particular embodiments, the inventors have found that for evaluating excrement event data a bandwidth h which is approximately 1 hour is advantageous.

Alternatively, the Kernel function may be another symmetric function having equal extension before and after the time mark, such as a uniform, a triangular, an Epanechikov, a quartic (biweight), a triweight, a tricube, or cosine function. The functions are well known for the skilled person and are therefore not explained in detail here.

In some cases an asymmetric Kernel function may be advantageous. For instance, before bedtime a Kernel function having an extension in time before the time mark may be useful, since in that way a wetness event occurring slightly after a time interval before bedtime may be associated to the time interval before bedtime instead of the time interval after bedtime.

Further, the method includes visually representing the number of associated excrement events for each time interval for visualising a pattern of excrement events based on the provided excrement data. The number of associated excrement events for each sequential time interval is arranged in form of a diagram or time diagram extending over the monitoring period, wherein each segment of the time diagram is corresponding to each time interval into which the monitoring period is divided and wherein the number of excrement events is associated with the corresponding segment. A time diagram is intended to mean a diagram which has a linear or circumferential form, wherein each segment is associated with a specified time interval.

In more detail, the method for monitoring or evaluating excrement data for seeking a pattern for a monitoring period includes:
associating each time interval with a segment of a diagram or time diagram;
calculating a number of excrement events corresponding to each time interval;
and associating the number of excrement events corresponding to each time interval with a corresponding segment of the diagram or time diagram.

According to the method, calculating the number of excrement events corresponding to each time interval includes calculating a sum of excrement events originating from each period, corresponding to the monitoring period, corresponding to each time interval over the total period for which data is provided. Suitably, the method includes correlating the time mark of each excrement event with a corresponding time interval prior to calculating the number of excrement events corresponding to each time interval. In the exemplary embodiment, the time intervals are predetermined, that is, the number of time intervals, the length of the time intervals, the sum of the length of the time intervals, and/or the alignment of the time series in relation to the monitoring period, etc. are predetermined. Further, predetermined series of time intervals are intended to be predetermined in respect of the number of time intervals, the length of the time intervals, the sum of the length of the time intervals, or the alignment of the time intervals in relation to the monitoring period, etc.

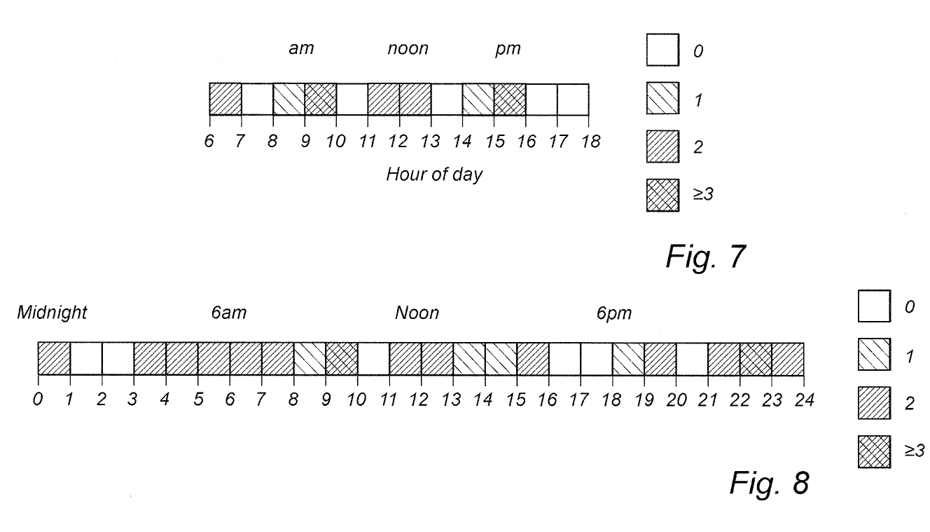

In the embodiments in FIGS. 7-9, the time intervals have equal lengths and are non-selectable by the user or care giver. That is, a person using the system, i.e. an operator or nurse, cannot select the length of the time intervals. Alternatively, the time intervals may be preselected or selected by the system. Still alternatively, the series of time intervals are determined by the method or the system adapted to perform the method.

In the embodiments in FIGS. 7-9, a sum of the length of said time intervals is shorter than the period of time corresponding to the period for which data is provided. Also, the sum of the length of said time intervals equals the length of the monitoring period.

The length of each of the predetermined time intervals is 20-150 minutes, 30-90 minutes, 45-75 minutes, or 55-65 minutes. In the embodiments in FIGS. 7-9, the time intervals have a length of 1 hour or 60 minutes.

Further, the measurement period, including all time periods for which excrement data is acquired or provided and each of which corresponds to the monitoring period, is predetermined, and has a length of two to seven times the monitoring period, the length of the measurement period is at least three times the monitoring period, or the length of the measurement period is substantially three times the monitoring period.

According to the embodiments in FIGS. 7-9, the number of associated excrement events for each time interval is associated with a graphical scheme applied to the segments of the diagram or time diagram. The graphical scheme may for instance be in form of rectangles having different heights or widths, wherein a wider or higher rectangle is associated with a higher number of excrement events. Other alternatives are a predetermined colour or shade scheme, or a colour or shade gradient, wherein a lighter colour or shade is associated with a lower number and a darker colour or shade with a higher number, or any other suitable graphical scheme, which makes the pattern easy to perceive. A colour or shade scheme may be in form of a colour or shade gradient or different density for one colour or shade, or different colours or shades for different number of events, etc.

As may be seen from FIGS. 7-9, the colour scheme of the exemplary embodiments are in form of a colour or shade gradient, wherein an increasing darkness is associated with an increasing number of associated excrement events.

In the exemplary embodiment in FIG. 7, the number of associated excrement events for each time interval is arranged in form of a linear time diagram extending over the monitoring period. The monitoring period of the data presented in FIG. 7 extends over a part of a day, from 6 to 18 if a 24-hour clock is used, which corresponds to 6 a.m. to 6 p.m. Each segment of the linear time diagram is associated with each of the provided time intervals. In the embodiment in FIG. 7, the length of each time interval is 1 hour and the intervals extend between 6 and 7, 7 and 8, etc. The number of excrement events associated with each interval is associated with corresponding associated segment. The graphical scheme is in form of a colour or shade gradient, such that the colour or shade saturation of the segments of the diagram associated with the intervals is proportional to the number of associated excrement events. That is, intervals with a low number of associated excrement events has a lower colour or shade saturation or a lighter colour or shade, and intervals with a high number of associated excrement events has a higher colour or shade saturation or a darker colour or shade. The excrement data includes data for a total measurement period, including the at least two different periods, covering three monitoring periods. Therefore, the colour gradient is adjusted for data covering three monitoring periods. For no events the colour or shade is white or none. For one event the colour or shade has lowest saturation or is lightest. For three or more events the colour or shade has highest saturation, or is darkest. For two events the colour or shade has higher saturation or is darker than for one event and has lower saturation or is lighter than for three events. In FIG. 7, the number of excrement events associated with the interval between 9 and 10 is 3. The number of excrement events associated with the intervals between 6 and 7, between 11 and 12, between 12 and 13, as well as between 15 and 16 is 2. The number of excrement events associated with the intervals between 8 and 9, as well as between 14 and 15 is 1. The number of excrement events associated with the intervals between 7 and 8, between 10 and 11, between 13 and 14, between 16 and 17, as well as between 17 and 18 is 0.

In the exemplary embodiment in FIG. 8, the number of associated excrement events for each time interval is arranged in form of a linear time diagram extending over the monitoring period. The monitoring period of the data presented in FIG. 8 extends over a whole 24 hour day, from 0 to 24 if a 24-hour clock is used, which corresponds to 0 a.m. to 12 μm. Each segment of the linear time diagram is associated with each of the provided time intervals. In the embodiment in FIG. 8, the length of each time interval is 1 hour as in FIG. 7 and the intervals extends between 0 and 1, 1 and 2, etc. The number of excrement events associated with each interval is associated with corresponding associated segment. The graphical scheme is in form of a colour or shade gradient, such that the colour saturation of the segments of the diagram associated with the intervals is proportional to the number of associated excrement events. That is, intervals with a low number of associated excrement events has a lower colour or shade saturation or a lighter colour or shade, and intervals with a high number of associated excrement events has a higher colour or shade saturation or a darker colour or shade. The excrement data includes data for a total measurement period covering three monitoring periods. Therefore, the colour or shade gradient is adjusted for data covering three monitoring periods. For no events the colour or shade is white or none. For one event the colour has lowest saturation or is lightest. For three or more events the colour or shade has highest saturation, or is darkest. For two events the colour or shade has higher saturation or is darker than for one event and has lower saturation or is lighter than for three events. In FIG. 8, the number of excrement events associated with the interval between 2 and 3, between 9 and 10, as well as between 22 and 23, is. The number of excrement events associated with the intervals for instance between 5 and 6, between 6 and 7, between 12 and 13, as well as between 15 and 16 is 2. The number of excrement events associated with the intervals between 8 and 9, between 13 and 14, between 14 and 15, as well as between 18 and 19, is 1. The number of excrement events associated with the intervals between 1 and 2, between 10 and 11, between 16 and 17, between 17 and 18, as well as between 20 and 21, is 0. As is clear, an unambiguous pattern is hard to find for the data in FIG. 8. Thus, the recommendation based on the method may be for some individuals to wear an absorbent product all the time, if there seems not to be a clear pattern.

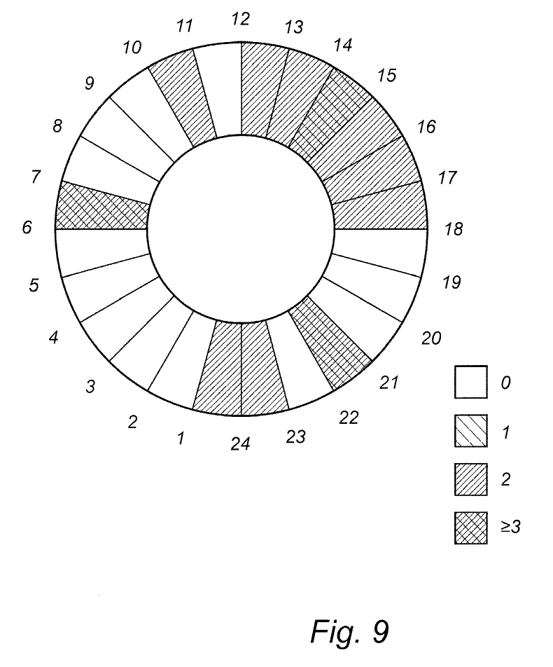

In the exemplary embodiment in FIG. 9, the number of associated excrement events for each time interval is arranged in form of a circular diagram or time diagram, which is an example of a circumferential diagram or time diagram according to the claims. The circular time diagram extends over the monitoring period, wherein each segment of the circular time diagram corresponds to each of the first time intervals. The start time and end time of the circular diagram coincide. In FIG. 9, the circular diagram is in form of a 24 hour clock diagram or clock chart. Alternatively, the circumferential diagram, or time diagram, have any other suitable form, such as quadratic, elliptic, etc. That is, the time diagram may be for instance quadratic or elliptic 24 hour clock or clock chart, or the diagram may be any other time diagram having a circumferential form suitable for illustrating the information.

Each segment of the circular time diagram corresponds to 55 to 65 minutes, especially, in FIG. 9, each segment corresponds to 1 hour. The number of associated excrement events for each time interval is associated with a graphical scheme which is applied to the segments of said time diagram. The graphical scheme is in form of a colour or shade gradient, associating an increasing darkness with an increasing number of associated excrement events. Generally, zero excrement events associated with a time interval is associated with white colour, or no colour, or no shade. Number of excrement events associated with a time interval being equal to or exceeding number of time periods corresponding to the monitoring period for which data is provided is associated with darkest colour or shade or highest colour or shade saturation. The number of excrement events associated with a time interval being equal to between one and one half of the number of time periods corresponding to the monitoring period for which data is provided has a slightly, but clearly visibly, higher colour or shade saturation, or a slightly darker or shade colour, than white or no colour or no shade. The number of excrement events associated with a time interval being equal to between one half of the number of time periods corresponding to the monitoring period for which data is provided and less than the number of time periods corresponding to the monitoring period for which data is provided is associated with a slightly, but clearly visibly, lower colour or shade saturation or a slightly lighter colour or shade than the darkest colour or shade.

In the embodiment in FIG. 9, this corresponds roughly to that the colour or shade applied to a segment associated with an interval associated with zero excrement events is white or none. The colour or shade applied to a segment associated with an interval associated with one excrement event has lowest colour saturation or is lightest besides white. The colour or shade applied to a segment associated with an interval associated with two excrement events has a slightly, but clearly visibly, higher colour or shade saturation or is slightly darker than the lightest colour or shade associated with one excrement event. And finally, the colour or shade applied to a segment associated with an interval associated with three or more excrement events has highest colour or shade saturation or is darkest. All of the colours or shades are naturally clearly distinguishable from each other without any additional vision aids in addition to those possibly normally used by the user using the invention.

According to an alternative embodiment, not shown in the figures, the diagram or time diagram may be in form of a histogram, wherein the size, that is, either width or height, of each rectangle is proportional against the number of events per time interval. Alternatively, instead of being in form of a colour or shade gradient or different density or saturations of one colour, the graphical scheme may be in from of different colours, or shades, for different number of events, or in from of symbols having different sizes, wherein an increasing size is indicative of a higher number of events, etc.

Further, the method includes visually representing the time diagram using suitable display means for visualising a pattern of events based on the provided data. The display means may be any suitable means such as a display, a handheld device, paper etc.

Embodiments of the invention also relate to a method for recommending toileting of a person associated with at least one absorbent article based on excrement data including the inventive method for analysing data and recommending toileting an interval prior to an interval for which the number of associated excrement events are equal to or larger than the number of time periods corresponding to the monitoring period for which data is provided.

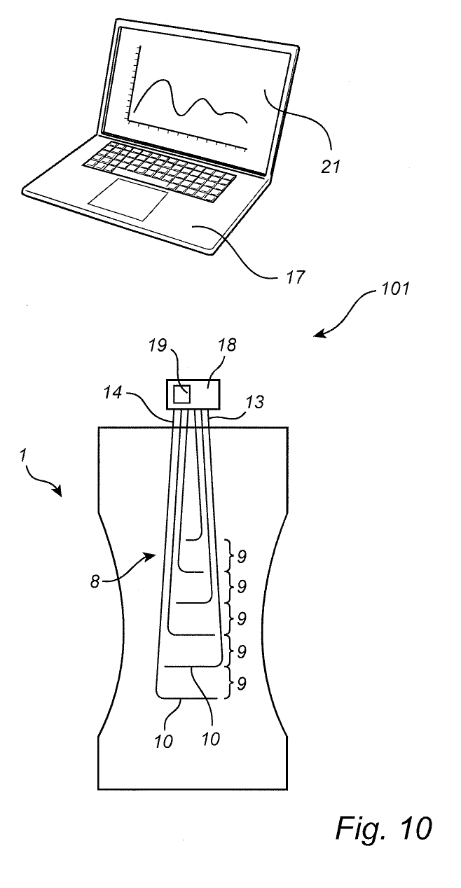

According to an embodiment of the invention, a system 101 for analysing excrement data is provided. FIG. 10 illustrates a system 101 for analysing an excrement data. The system 101 includes an absorbent article and a data processing unit 17, arranged separate from the absorbent article 1. In FIG. 10 the absorbent article is shown in form of an adult incontinence product, i.e. a diaper 1. The principles are, however, applicable to other absorbent articles such as baby or toddler diapers, sanitary towels or other known absorbent articles. The diaper 1, which is illustrated in FIG. 10, is an example of a conventional diaper except for the presence of a sensor 5 adapted to acquire excrement data. Further, the absorbent article 1 includes a control unit contact area 13 to which a control unit 18 is to be connected in order to activate each of the detection zones 9 to get a supply of excrement reading. The contact area 13 is located at a laterally central front waist region of the absorbent article 1. The contact area 13 includes a plurality of electrical contacts 14 for making electrical contact with corresponding contacts on the control unit 18. Each conductive path 10 is connected to a respective electrical contact 14 by way of a respective electrically conductive lead. The combination of a given contact 14 and a conductive path 10 may be formed of a unitary structure (such as a conductive thread).

Further, the exemplary embodiment of the system 101 in FIG. 10 includes a control unit 18. The control unit 18 includes contacts to engage with the contacts 14 of the protruding tab of tape of the absorbent article 1. The control unit 18 includes a memory card to provide hard memory, a memory buffer, a measurement circuit for measuring an electrical property, a clock, a battery, a wireless transmitter, and a processor 19. The battery is used to power operation of all of the components of the control unit 18.

The measurement circuit is configured to regularly apply a potential between adjacent pairs of conductive paths 10 of the absorbent article 1 and measure or indicate the impedance there between.

The processor 19 of the control unit 18 may be configured to take the measurement data from the measurement circuit and store it in the buffer until a sequence of a set of measurement data for all of the pairs is stored in the buffer. The processor is further configured to store a clock reading with each set measurement data. The storage of this set of data is repeated regularly (e.g. every second). The processor is configured to transfer the data from the buffer memory to a remote memory unit, such as a hard memory of some kind of central computer, for remotely recording data. Alternatively, the data may be written into a memory card, which is removable so that the stored data may be accessed by remotely located analysis software. Still alternatively, the stored data may be accessed by a cable, a USC connection or the like. In such instances, other implementations of the hard memory than a memory card may be used.

The data processing unit 17 is located in some kind of central computer and includes a microcomputer and software for performing at least a portion of the method according to an embodiment of the invention. The data processing unit 17 is used to process the stored excrement data into a useful form for performing detecting and for evaluating excrement data according to the method described above.

A receiver arranged in the central computer is used to retrieve the data transmitted by the transmitter of the control unit 18. Thereafter, the excrement data is acquired or received by the data processing unit 17. The data processing unit 17 may take the excrement data for each of the detection zones 9 from the memory and detect excrement events. Alternatively, the data processing unit 17 is configured to receive sets of data indicating excrement events into the absorbent article. Further, the data processing unit 17 is adapted to perform/execute the inventive method for analysing excrement data described above. That is, the processing unit provides a series of time intervals together substantially extending over the monitoring period; and associates/correlates the time marks of each excrement event with a corresponding time interval. Further, data processing unit is further configured to estimate a pattern of excrement events based on the association/correlation of the time mark of each excrement event with a corresponding time interval. The data processing unit 17 includes means for estimating a toileting schedule for a person associated with said absorbent article 1, based on said estimated pattern of excrement events.

Further, the processing unit 17 performs embodiments of the inventive method for monitoring or evaluating excrement data described above. That is, the processing unit 17 provides a series of time intervals together substantially extending over the monitoring period, as is described above; associates each time interval with a segment of a diagram or time diagram. The time diagram extends substantially over the monitoring period. The data processing unit 17 calculates a number of excrement events corresponding to each time interval; and arranges the number of excrement events corresponding to each time interval into a corresponding segment of said time diagram. The processing unit 17 is further adapted to calculate the number of excrement events corresponding to each time interval by calculating a sum of excrement events, which events correspond to each time interval and originates from each at least two periods for which data is provided, and which corresponds to the monitoring period. Suitably, the data processing unit 17 is adapted to correlate the time mark of each excrement event with a corresponding time interval prior to calculating the number of excrement events corresponding to each time interval.

Further, the data processing unit 17 is adapted to associate a number of associated excrement events for each time interval with a graphical scheme and to apply the scheme to the segments of the time diagram or diagram.

The data processing unit 17 is further an example of means for estimating a toileting schedule for a person associated with the absorbent article 1 based on the estimated pattern of excrement events.

Instead of being integrated in a central computer, the data processing unit 17 may be integrated in a cell phone, some kind of handheld computer, etc. Still alternatively, instead of including both a processing unit 19 integrated in the control unit 18 and a remote data processing unit 17 integrated in the central computer, the system may include the single data processing unit 17, 19 integrated into the control unit 18 alone or in the computer 2 alone. In such a case the single data processing unit 17, 19 of the control unit 18 or computer 2 is adapted to acquire data as well as to process it.

Further, the exemplary embodiment in FIG. 10 includes a display unit 21, which is an example of output means for visually representing the number of associated/correlated time marks of each excrement event for each corresponding time interval for visualising a pattern of excrement events based on the provided excrement data. The display unit 21 is connected or connectable to the data processing unit 17 and arranged to display the result of the inventive method, such as the excrement data analysed according to an embodiment of the inventive method such as a pattern of excrement events is apparent as well as a visual representation of the number of excrement events associated with each interval according to the inventive method.

The diaper 1, which is illustrated in FIG. 10, is an example of a conventional diaper except for the presence of a wetness or liquid discharge sensor 8 including a plurality of wetness detection zones 9 (in this specific example, there are five wetness detection zones 9). The wetness sensor 8 is adapted to generate an electrical output signal representative of a wetness state or degree of wetness of the absorbent core of the diaper 1. The wetness sensor 8 includes several zones or segments 9 and is responsive to a change in an electrical property in the core of the absorbent article and includes conductive material arranged in contact with the core of the absorbent article. The electrical property is conductance, resistance, or other electrical properties linked to these. In the exemplary diaper 1 in FIG. 10, each detection zone 9 includes first and second electrically conductive paths 10 (in the form of electrically conductive threads, or other electrically conductive material) that are longitudinally spaced from one another along a longitudinal axis of the absorbent article 1. The conductive paths 10 are in physical and electrical contact with the absorbent core. The end detection zones 9 share a conductive path 10 with an adjacent zone, while the middle detection zones 9 share both conductive paths 10 with their adjacent detection zones 9.

The sensor is a disposable sensor and intended to be used once and disposed together with the used absorbent article. Further, the sensor is arranged inaccessibly for a user in the absorbent article and is integrated into the absorbent article. That is, the sensor is not accessible to the user without at least partly breaking the absorbent article.

The scope of the invention according to the claims is not limited to the diaper 1 described above or the wetness sensor 8 described above. The principles of the present invention are, however, applicable to other absorbent articles such as baby or toddler diapers, sanitary towels or other known absorbent articles. Further, the principles of the present invention are applicable to other suitable sensors 8 including one detection zone 9 or a plurality of detection zones 9 as well. Alternatively, the sensor according to the claims may be any other suitable sensor which is responsive to an excrement event in the absorbent article, such as another wetness or liquid discharge sensor responsive to a change in an electrical property in the absorbent article, including conductive material, a temperature sensor, a gas sensor etc. Still alternatively, the sensor may be reusable and adapted to be attached to and detached from the absorbent article.

It is also useful to determine the length of time, particularly in a system of absorbent articles, before a first excrement event. The data begins at time zero when the control unit is first contacted with the contacts of the absorbent article. The system is thus configured to determine the length of time from when data is first recorded for that absorbent article to when the impedance measurement shows that a first excrement event has occurred. Further, the time between the first supply of excrement and any subsequent excrement events is recorded. The time data is stored in the excrement data and the data processing unit is configured to evaluate the data according to the inventive method.

Embodiments of the invention also relate to a system for recommending toileting based on excrement data including the system for evaluating data and means for recommending toileting an interval prior to an interval for which the number of associated excrement events are equal to or larger than the number of monitoring periods for which data is acquired or provided. According to at least an embodiment, the time interval for which toileting is recommended may be indicated in the diagram by a predetermined symbol.

Even though the invention has been described with reference to specific exemplifying embodiments thereof, many different alterations, modifications and the like will become apparent for those skilled in the art. For example, additionally, variations to the disclosed embodiments may be understood and effected by the skilled addressee in practicing the claimed invention, from a study of the drawings, the disclosure, and the appended claims. In the claims, the word “comprising” does not exclude other elements or steps, and the indefinite article “a” or “an” does not exclude a plurality. A single unit may fulfil the functions of several items recited in the claims. The mere fact that certain measures are recited in mutually different dependent claims does not indicate that a combination of these measured cannot be used to advantage.

1: A method for monitoring excrement data for seeking a pattern for a monitoring period, wherein said excrement data is associated with at least one absorbent article and a person wearing said at least one absorbent article, the method comprising:
acquiring excrement data comprising excrement data for at least two different time periods, each substantially corresponding to the monitoring period for which a pattern of events is sought, wherein said excrement data comprises information of excrement supply into said at least one associated absorbent article in form of excrement events, and wherein each excrement event has an associated time mark or associated time information from which an associated time mark may be derived;
providing a series of sequential non-overlapping time intervals, together substantially extending over the monitoring period;
associating each time interval with a segment of a diagram;
calculating a number of excrement events corresponding to each time interval for the at least two time periods for which data is acquired; and
associating the number of excrement events corresponding to each time interval with a corresponding segment of said diagram.

2: The method according to claim 1, further comprising combining the excrement data of the at least two different time periods to aggregated data extending over the monitoring period, covering the data for the at least two different time periods.

3: The method according to claim 2, wherein combining the data to excrement data of the at least two different time periods to aggregated data comprises superposing the excrement data of the at least two different time periods such that each event of the superposed excrement data is distinguishable and such that the hours of the superposed sets of data coincide with the hours of the monitoring period.

4: The method according to claim 1, wherein said excrement data is in form of absorbent state data and an excrement event is indicated by a change of the absorbent state of the absorbent article.

5: The method according to claim 1, wherein said excrement data is in form of electrical property data and an excrement event is indicated by a change of the electrical property of the absorbent article.

6: The method according to claim 1, wherein calculating the number of excrement events corresponding to each time interval comprises calculating a sum of excrement events corresponding to each time interval, wherein the sum of excrement events is a sum of the number of excrement events of the aggregated data corresponding to each time interval, and wherein associating the number of excrement events corresponding to each time interval with a corresponding segment of said diagram comprises associating said sum of excrement events with a corresponding segment of said diagram

7: The method according to claim 1, further comprising correlating the time mark of each excrement event with a corresponding time interval prior to calculating the number of excrement events corresponding to each time interval.

8: The method according to claim 1, wherein said time intervals are pre-determined.

9: The method according to claim 8, wherein the length of each of said predetermined time intervals is 20 minutes to 150 minutes.

10: The method according to claim 1, wherein the total period, comprising the at least two different time periods for which excrement data is acquired, is predetermined, and has a length that corresponds to two to seven times the monitoring period.

11: The method according to claim 1, wherein the number of associated excrement events for each time interval is associated with a correlating graphical scheme applied to the segments of said diagram.

12: The method according to claim 1, wherein the number of associated excrement events for each time interval is associated with a correlating colour scheme applied to the segments of said diagram.

13: The method according to claim 12, wherein the colour scheme is in form of a colour gradient.

14: The method according to claim 1, claims, wherein toileting of the person associated with the excrement data is recommended during a time interval prior to an interval for which the number of associated excrement events is equal to or larger than the number of the time periods corresponding to the monitoring period corresponding to time periods for which data is acquired.

15: The method according to claim 14, wherein the time interval for which toileting is recommended is indicated in the diagram by a predetermined symbol.

16: The method according to claim 1, wherein the number of associated excrement events for each time interval is arranged in form of a linear diagram.

17: The method according to claim 1, wherein the number of associated excrement events for each time interval is arranged in form of a circumferential diagram extending substantially over the monitoring period, wherein each segment of the circumferential diagram corresponds to each of the time intervals.

18: The method according to claim 1, wherein the number of associated excrement events for each time interval is arranged in form of a circular diagram extending substantially over the monitoring period, wherein each segment of the circular diagram corresponds to each of the time intervals.

19: The method according to claim 17, wherein the circumferential diagram is in form of a circular diagram.

20: The method according to claim 17, wherein each segment of the circumferential diagram is corresponding to 55 to 65 minutes.

21: The method according to claim 1, further comprising visually representing the diagram for visualising a pattern of events based on the acquired data.

22: A processing unit adapted to monitor excrement supply data, said processing unit adapted to process an output signal generated by a sensor associated with an absorbent article, wherein said processing unit is adapted to:
acquire excrement data comprising excrement data for at least two different time periods, each substantially corresponding to the monitoring period for which a pattern of events is sought, wherein said excrement data comprises information of excrement supply into an absorbent article in form of excrement events, and wherein each excrement event has an associated time mark or associated time information from which an associated time mark may be derived;
provide a series of sequential non-overlapping time intervals, together substantially extending over the monitoring period;
associate each time interval with a segment of a diagram, which diagram substantially extends over the monitoring period;
calculate a number of excrement events corresponding to each time interval; and
arrange said number of excrement events corresponding to each time interval into a corresponding segment of said diagram.

23: The processing unit according to claim 22, further adapted to calculate the number of excrement events corresponding to each time interval by calculating a sum of excrement events originating from each different time period corresponding to the monitoring period corresponding to each time interval.

24: The processing unit according to claim 22, further adapted to correlate the time mark of each excrement event with a corresponding time interval prior to calculating the number of excrement events corresponding to each time interval.

25: The processing unit according to claim 22, wherein said processing unit is further adapted to associate a number of associated excrement events for each time interval with a graphical scheme and to apply said scheme to the segments of said diagram.

26. (canceled)

27: A system for monitoring excrement data, comprising:
a sensor associated with an absorbent article, which is arranged to generate an output signal representative of the excrement supply status of said absorbent article over at least two different time periods, and
the processing unit according to claim 22.

28: The system according to claim 27, further comprising output means for visually representing the number of correlated time marks of excrement events for each corresponding time interval for visualising a pattern of events based on the excrement data.

29. (canceled)
